(19) United States
(12) Patent Application Publication
(10) Pub. No.: US 2019/0133516 A1
(43) Pub. Date: May 9, 2019

(54) PHYSIOLOGICAL MONITOR FOR MONITORING PATIENTS UNDERGOING HEMODIALYSIS

(71) Applicant: TOSENSE, INC., SAN DIEGO, CA (US)

(72) Inventors: Matthew BANET, SAN DIEGO, CA (US); Marshal Singh DHILLON, SAN DIEGO, CA (US); Susan Meeks PEDE, ENCINITAS, CA (US); Lauren Nicole Miller HAYWARD, SAN DIEGO, CA (US); Mark Singh DHILLON, SAN DIEGO, CA (US); Jeffrey KLEIN, SAN DIEGO, CA (US); Derek STAINER, SAN DIEGO, CA (US); R. Craig BROADBOOKS, CARDIFF, CA (US)

(73) Assignee: TOSENSE, INC., SAN DIEGO, CA (US)

(21) Appl. No.: 16/307,909

(22) PCT Filed: Jun. 6, 2017
(86) PCT No.: PCT/US2017/036221

§ 371 Date: Dec. 6, 2018

Related U.S. Application Data
(60) Provisional application No. 62/346,410, filed on Jun. 6, 2016.

(51) Int. Cl. A61B 5/00; A61B 5/0205; A61M 1/14

(52) U.S. Cl. CPC A61B 5/4836; A61B 5/6823; A61B 5/0205; A61B 5/02416; A61B 5/7225; A61M 1/14; A61B 5/0002

(57) ABSTRACT

The invention provides a system for characterizing a patient undergoing hemodialysis, featuring: 1) a body-worn biometric sensor, worn on a single location of the patient, and featuring: i) sensing elements for measuring electrocardiogram (ECG), thoracic bio-impedance (TBI), photoplethysmogram (PPG), and phonocardiogram (PCG) waveforms; ii) a processor for collectively analyzing the ECG, TBI, PPG, and PCG waveforms to determine a set of physiological parameters; and iii) a first wireless transceiver configured to transmit the set of physiological parameters; 2) a gateway system comprising a second wireless transceiver configured to receive the set of physiological parameters; and 3) a data-analytics system configured to analyze the set of physiological parameters to determine the patient's status.

### CROSS-REFERENCE TO RELATED APPLICATIONS

This application claims the benefit of U.S. Provisional Application No. 62/346,410 filed Jun. 6, 2016, which is hereby incorporated in its entirety including all tables, figures, and claims.

### BACKGROUND AND FIELD OF THE INVENTION

### Field of the Invention

The invention relates to the use of sensors that measure physiological signals from patients undergoing medical treatment, e.g. hemodialysis.

### General Background

There are a number of physiological parameters that can be assessed by measuring physiological or physiologically influenced biometric signals from a patient. Some signals, such as electrocardiogram (ECG), thoracic bio-impedance (TBI), photoplethysmogram (PPG), and phonocardiogram (PCG) waveforms, are measured with sensors (e.g. electrodes, optics, microphones) that attach directly to the patient's skin. Processing of these waveforms yields parameters such as heart rate (HR), respiration rate (RR), heart rate variability (HRV), pulse oximetry (SpO2), stroke volume (SV), cardiac output (CO), and parameters related to thoracic fluids, e.g. thoracic fluid content (TFC). Certain physiological conditions can be identified from these parameters when they are obtained at a single point in time; others require assessment over some period of time to identify trends in the parameters. In both cases, it is important to obtain the parameters consistently and with high repeatability and accuracy.

Measuring some physiological parameters does not require a high degree of precision and/or consistency in the body location at which a measurement is taken. As a simple example, measuring a patient's temperature (TEMP) is frequently performed with an oral thermometer that is simply placed somewhere under the tongue. Here, the exact placement of the thermometer does not have a big impact on the measured temperature value. Likewise, parameters that depend on time-dependent features in waveforms, such as HR, which depends on the time-dependent variation of R-R intervals in the ECG waveforms, are relatively insensitive to sensor positioning. In this case, the R-R intervals show almost no variation with positioning of electrodes on the patient's thoracic cavity. Systolic (SYS) and diastolic (DIA) blood pressure (BP), in contrast, shows some sensitivity to measurement location. When measured with a sphygmomanometer, BP values are relatively insensitive to the general alignment of the cuff over the brachial artery, but will vary when measured at other locations on the body, such as the wrist, thigh, finger, or even the opposing arm. Likewise, measuring amplitude-dependent features in waveforms, such as TFC, will strongly depend on the positioning of electrodes. In this case, the value of TFC relates to the impedance measured between the electrodes, and this in turn will vary with the electrodes' placement. Deviation in day-to-day placement of electrodes can result in measurement errors, particularly when trends of the measured parameters are extracted. This, in turn, can lead to misinformation, nullify the value of such measurements, and thus negatively impact treatment.

### Known Devices and Relevant Physiology

Medical devices that measure time-dependent ECG and TBI waveforms from patients typically connect through cables or lead wires to disposable electrodes adhered at various locations on a patient's body. Analog circuits within a given device, which are typically located remote from the patient's body in the device, process the signals to generate the waveforms. With further analysis, such waveforms yield parameters such as HR, TFC, SV, CO, and RR.

Disposable electrodes that measure ECG and TBI waveforms are typically worn on the patient's chest or legs and include: i) a conductive hydrogel that contacts the patient; ii) a Ag/AgCl-coated eyelet that contacts the hydrogel; iii) a conductive metal post that connects the eyelet to a lead wire or cable extending from the device; and iv) an adhesive backing that adheres the electrode to the patient. Some devices that measure ECG and TBI waveforms are worn entirely on the patient's body. These devices have been improved to feature simple, patch-type systems that include both analog and digital electronics connected directly to underlying electrodes. Such devices are typically prescribed for relatively short periods of time, e.g. for a time period ranging from a few days to several weeks. They are typically wireless, and include technologies such as Bluetooth® transceivers to transmit information over a short range to a second device, which typically includes a cellular radio to transmit the information to a web-based system.

5. Patients with End-Stage Renal Disease and Congestive Heart Failure

Assessing TFC, weight, and hydration status is important in diagnosing and managing many diseases. For example, end-stage renal disease (ESRD) occurs when a patient's kidneys are no longer able to work at a level needed for day-to-day life. The disease is most commonly caused by diabetes and high blood pressure, and is characterized by a gradual increase in fluids throughout the body, along with swings in SYS and DIA. Patients suffering from ESRD typically require hemodialysis (including ultrafiltration) to remove excess fluids. Thus to characterize ESRD, accurate measurement of TFC can eliminate the need for empirical clinical estimations that often lead to over-removal or under-removal of fluid during hemodialysis, thereby preventing hemodynamic instability and hypotensive episodes (Anand et al., “Monitoring Changes in Fluid Status With a Wireless Multisensor Monitor: Results From the Fluid Removal During Adherent Renal Monitoring (FARM) Study,” Congest Heart Fail. 2012; 18:32-36).

There are two main types of dialysis: ‘hemodialysis’ and ‘peritoneal dialysis’. Hemodialysis is typically performed three times a week, 52 weeks a year, in over 400,000 people in the United States. It uses a technique called ultrafiltration to remove excess waste products and water from the patient's blood. Peritoneal dialysis uses a fluid that is placed into the patient's abdominal cavity through a special plastic tube to remove excess waste products and fluid from the body. During hemodialysis, blood passes from the patient's body through a filter (a ‘dialysis membrane’) into a dialysis machine. For this procedure, the patient has a specialized plastic tube placed between an artery and a vein in their arm or leg (called a ‘gortex graft’). Sometimes a direct connection is made between an artery and a vein in the arm. This procedure is called a ‘Cimino fistula’. Needles are then placed in the graft or fistula, and blood passes to the dialysis machine, through the filter, and back to the patient. If the patient requires dialysis before a graft or a fistula is placed, a large diameter catheter is typically placed directly into a large vein in the neck or leg in order to perform dialysis. In the dialysis machine, a solution on the other side of the filter receives the waste products from the patient.

Peritoneal dialysis uses the patient's own body tissues inside of the abdominal cavity to act as the filter. The abdominal cavity is lined with a special membrane, called the ‘peritoneal membrane’. A plastic tube called a ‘peritoneal dialysis catheter’ is placed through the abdominal wall into the abdominal cavity. A special fluid is then flushed into the abdominal cavity and washes around the intestines. The peritoneal membrane acts as a filter between this fluid and the blood stream. By using different types of solutions, waste products and excess water can be removed from the body through this process.

Since each hemodialysis procedure involves removal of large volumes of fluid, it is not uncommon for a patient to experience intradialytic symptomatic hypotension, muscle cramps, nausea, vomiting, and other uncomfortable conditions. The current standard technique for measuring interdialytic fluid gain in a dialysis patient is to compare their current weight against a known ‘dry weight’, which is essentially estimated from the patient's weight at the beginning of the hemodialysis session. However, such a weight can be erroneously influenced by a number of factors not related to fluid retention. An accurate measurement of the rate of fluid removal from the patient has the potential to minimize the above-described intradialytic complications, and could also be used to estimate a progression towards the patient's ideal dry weight.

Many ESRD patients also suffer from congestive heart failure (CHF), a complicated disease that is typically monitored using a “constellation” of physiological factors, i.e., fluid status (e.g. TFC), vital signs (i.e. HR, RR, TEMP, SYS, DIA, and SpO2), and hemodynamic parameters (e.g. CO, SV). Accurate measurement of these parameters can aid in managing patients, particularly for dispersing diuretic medications, thereby reducing expensive hospital readmissions (Packer et al., “Utility of Impedance Cardiography for the Identification of Short-Term Risk of Clinical Decompensation in Stable Patients With Chronic Heart Failure,” J Am Coll Cardiol 2006; 47:2245-52).

CHF is a particular type of heart failure (HF), a chronic disease driven by complex pathophysiology. This condition occurs when SV and CO are insufficient in adequately perfusing the kidneys and lungs. Causes of HF are well known and typically include coronary heart disease, diabetes, hypertension, obesity, smoking, and valvular heart disease. In systolic HF, ejection fraction (EF) can be diminished (<50%), whereas in diastolic HF this parameter is typically normal (>65%). The common signifying characteristic of both forms of heart failure is time-dependent elevation of the pressure within the left atrium at the end of its contraction cycle, or left ventricular end-diastolic pressure (LVEDP). Chronic elevation of LVEDP causes transudation of fluid from the pulmonary veins into the lungs, resulting in shortness of breath (dyspnea), rapid breathing (tachypnea), and fatigue with exertion due to the mismatch of oxygen delivery and oxygen demand throughout the body. Thus, early compensatory mechanisms for HF that can be detected fairly easily include increased RR and HR.

As CO is compromised, the kidneys respond with decreased filtration capabilities, thus driving retention of sodium and water, and leading to an increase in intravascular volume. As the LVEDP rises, pulmonary venous congestion worsens. Body weight increases incrementally, and fluids may shift into the lower extremities. Medications for HF are designed to interrupt the kidneys' hormonal responses to diminished perfusion, and they also work to help excrete excess sodium and water from the body. However, an extremely delicate balance between these two biological treatment modalities needs to be maintained, since an increase in BP (which relates to afterload) or fluid retention (which relates to preload), or a significant change in HR due to a tachyarrhythmia, can lead to decompensated HF. Unfortunately, this condition is often unresponsive to oral medications. In that situation, admission to a hospital is often necessary for intravenous diuretic therapy.

In medical centers, HF is typically detected using Doppler/ultrasound, which measures parameters such as SV, CO, and EF. In the home environment, on the other hand, gradual weight gain measured with a simple weight scale is one method to indicate CHF. However, this parameter is typically not sensitive enough to detect the early onset of CHF—a particularly important time when the condition may be ameliorated by a change in medication or diet.

SV is the mathematical difference between left ventricular end-diastolic volume (EDV) and end-systolic volume (ESV) and represents the volume of blood ejected by the left ventricle with each heartbeat; a typical value is about 70-100 mL. EF relates to EDV and ESV as described below in Equation 1:

$$EF = \frac{SV}{EDV} = \frac{EDV - ESV}{EDV} \qquad ( 1 )$$

SV measured by bio-impedance is typically using an equation similar to the Bernstein equation, shown below in Eqn. 2. The Bernstein equation is described in more detail in the following reference, the contents of which are incorporated herein by reference: Bernstein, D. P., and H. J. M. Lemmens. “Stroke volume equation for impedance cardiography.” Medical and Biological Engineering and Computing 43.4 (2005): 443-450.

$$V = Vc \times Mc \times LVET \times \sqrt{\frac{( \frac{dZ ( t )}{dt} ) max}{{Z}_{0}}} \qquad ( 2 )$$

In Eqn. 2, Z₀ is calculated as the average value of a DC component of the TBI waveform. A volume conductor—Vc—represents an empirically derived, patient-specific constant related to the patient's blood volume. It typically depends on the patient's weight and gender. Mc is also an empirically derived parameter that is related to the morphology of a heartbeat-induced ‘cardiac pulse’ within an AC component of the TBI waveforms. Left ventricular ejection time (LVET) indicates a time separating the opening and closing of the aortic valve. This parameter is typically estimated directly from the cardiac pulse, or from the patient's current HR using a formula called Weissler's regression, shown below in Eqn. 3:

LVET (sec)=0.413−0.0017×HR (beats/min) (3)

CO is the average, time-dependent volume of blood ejected from the left ventricle into the aorta. It indicates how efficiently a patient's heart pumps blood through the arterial tree; a typical value is about 5-7 L/min. Eqn. 4 below shows CO as the product of HR and SV:

CO=SV×HR (4)

CHF patients, and in particular those suffering from systolic HF, may receive implanted devices such as pacemakers and/or implantable cardioverter-defibrillators to increase EF and blood flow throughout the body. These devices may include circuitry and algorithms to measure the electrical impedance between different leads of the device. As thoracic fluid increases in the CHF patient, the impedance typically is reduced. Thus, this parameter, when read by an interrogating device placed outside the patient's body, can indicate the onset of heart failure.

6. Costs Associated with ESRD and CHR

CHF and ESRD affect, respectively, about 5.3 million and 3 million Americans, resulting in annual healthcare costs estimated at $45 billion for CHF and $35 billion for ESRD. CHF patients account for approximately 43% of annual Medicare expenditures, which is more than the combined expenditures for all types of cancer. Somewhat disconcertingly, roughly $17 billion of this is attributed to hospital readmissions. CHF is also the leading cause of mortality for patients with ESRD, and this demographic costs Medicare nearly $90,000/patient annually. Thus, there understandably exists a profound financial incentive to keep patients suffering from these diseases out of the hospital. Starting in 2012, U.S. hospitals have been penalized for above-normal readmission rates. Currently, the penalty has a cap of 1% of payments, growing to over 3% in the next three years.

Of some promise, however, is the fact that CHF-related hospital readmissions can be reduced when clinicians have access to detailed information that allows them to remotely titrate medications, monitor diet, and promote exercise. In fact, Medicare has estimated that 75% of all patients with ESRD and/or CHF could potentially avoid hospital readmissions if treated by simple, effective programs.

Thus, with the aim of identifying precursors to conditions such as CHF and ESRD, physicians can prescribe monitoring solutions to patients living at home. Typically such solutions include multiple, standard medical devices, e.g. blood pressure cuffs, weight scales, and pulse oximeters. In certain cases, patients use these devices daily and in a sequential manner, i.e. one device at a time. The patient then calls a central call center to relay their measured parameters. In more advanced systems, the devices are still used in a sequential manner, but automatically connect through a short-range wireless link (e.g. Bluetooth®) to a “hub”, which then forwards the information off to a call center. Often the hub features a simple user interface that poses basic questions to the patient, e.g. questions concerning their diet, how they are feeling, and whether or not medications were taken.

### SUMMARY OF THE INVENTION

In view of the foregoing, it would be beneficial to improve the monitoring of patients during hemodialysis and similar therapies. A sensor according to the invention, which facilitates monitoring a patient suffering from ESRD, HF, CHF, cardiac arrhythmias, and other diseases in both home and clinical environments, could achieve this goal. The sensor is worn like a patch or conventional necklace, and features a mechanical mechanism that ensures consistent placement when used on a daily basis, thereby improving the repeatability and reproducibility of its measurements. Additionally, the sensor makes simultaneous measurements of multiple parameters, and thus obviates the need to use multiple devices. Both of these features may improve patient compliance.

More particularly, in one aspect the invention features a neck-worn sensor that measures the following parameters from a patient: HR, PR, SpO2, RR, BP, TEMP, TFC, SV, CO, and a set of parameters sensitive to blood pressure and systemic vascular resistance called pulse arrival time (PAT) and vascular transit time (VTT). From SV, a first algorithm employing a linear model can estimate the patient's pulse pressure (PP). And from PP and PAT, VTT, and related parameters, a second algorithm can estimate SYS and DIA. Thus, the sensor, acting alone, can measure all five vital signs (HR/PR, SpO2, RR, TEMP, and SYS/DIA) along with hemodynamic parameters (SV, CO, TFC). Trends in some of these parameters may predict the onset of adverse events that occur, e.g., during dialysis.

The sensor also includes a motion-detecting accelerometer, from which it can determine motion-related parameters such as posture, degree of motion, activity level, respiratory-induced heaving of the chest, and falls. Such parameters could determine, for example, a patient's posture or movement during a dialysis treatment; this may impact the progression towards cramping and nausea. The sensor can operate additional algorithms to process the motion-related parameters to measure vital signs and hemodynamic parameters when motion is minimized and below a pre-determined threshold, thereby reducing artifacts. Moreover, the sensor estimates motion-related parameters such as posture to improve the accuracy of calculations for vital signs and hemodynamic parameters.

Disposable electrodes attach directly to the sensor to secure it in close proximity to the patient's body without bothersome cables. In particular, the electrodes are typically provided in adhesive patches, with each electrode patch containing two electrode regions to measure ECG and TBI waveforms. The patches easily connect (and disconnect) to circuit boards contained within the sensor by means of magnets that are electrically connected to the circuit boards to provide signal-conducting electrical couplings. Prior to use, the electrodes are simply held near the circuit boards, and magnetic attraction causes the electrode patches to snap into proper position, thereby ensuring proper positioning of the electrodes on the patient's body.

Using light-emitting diodes operating in the red (e.g. 660 nm) and infrared (e.g. 900 nm) spectral regions, the sensor measures SpO2 and PR by pressing lightly against capillary beds in the patient's chest. Operating with reflection-mode optics, the sensor measures PPG waveforms with both red and infrared wavelengths. SpO2 is processed from alternating and static components of these waveforms. PR, in turn, can be calculated from neighboring pulses, typically from the PPG waveform generated with infrared light, as this typically has a relatively high signal-to-noise ratio.

All analog and digital electronics associated with these various measurements are directly integrated into the sensor. This means that a single, unobtrusive component—typically shaped like a piece of conventional jewelry instead of a bulky medical device—measures a robust set of parameters that can characterize a patient using both one-time and continuous measurements. Measurements can take place over just a few minutes or several hours, and can be made in medical facilities and at home. The sensor includes a simple LED in its base (i.e. sensing) portion, which is located near the center of the chest when worn by the patient. The sensor also includes a wireless transmitter (operating Bluetooth® and/or 802.11a/b/g/n) than sends data to, e.g., a gateway device. This can be, for example, a conventional mobile device (e.g. cellular telephone, tablet computer, desktop/laptop computer, or plug-in hub).

The sensor measures all of the above-mentioned properties while featuring a comfortable, easy-to-wear form factor. It is lightweight (about 100 grams) and battery-powered. During use, it simply drapes around the patient's neck, where the disposable electrodes hold it in place, as described in more detail below. Flexible, conductive elements resembling strands or cables in a conventional necklace power on the sensor, hold it in place, and also ensure that it is consistently positioned when used on a daily basis. Moreover, the patient's neck is a location that is unobtrusive, comfortable, removed from the hands, and able to bear the weight of the sensor without being noticeable to the patient. The neck and thoracic cavity are also relatively free of motion compared to appendages such as the hands and fingers, and thus a sensor affixed to the neck region minimizes motion-related artifacts. Moreover, such artifacts are compensated for, to some degree, by the accelerometer within the sensor. And because the sensor resembles jewelry (e.g., a necklace) and is therefore considerably less noticeable or obtrusive than various prior-art devices, emotional discomfort over wearing a medical device over an extended period of time is reduced, thereby fostering long-term patient compliance with a monitoring regimen.

Given the above, in one aspect, the invention provides a system for characterizing a patient undergoing dialysis, comprising: 1) a body-worn biometric sensor, worn on a single location of the patient, and featuring: i) sensing elements for measuring ECG, TBI, PPG, and PCG waveforms; ii) a processor for collectively analyzing the ECG, TBI, PPG, and PCG waveforms to determine a set of physiological parameters; and iii) a first wireless transceiver configured to transmit the set of physiological parameters; 2) a gateway system comprising a second wireless transceiver configured to receive the set of physiological parameters; and 3) a data-analytics system configured to analyze the set of physiological parameters to determine the patient's status.

In another aspect, the invention provides a system for estimating a ‘dry weight’ value of a patient undergoing a dialysis session. Here, the term dry weight represents what the patient should weigh without fluid built up because of kidney failure. The system includes: 1) a body-worn biometric sensor, similar to that described above, that measures TBI waveforms; 2) a processor for collectively analyzing the TBI waveforms to determine a fluid value of the patient, and then estimating the patient's dry weight value by analyzing the fluid value and a value of the patient's weight before the hemodialysis session begins. Here, the fluid value relates to a volume of fluid in the patient's chest. The fluid is made up of mostly water and has a density near 1 gram/cm³. Using this relationship, the fluid removed can be associated with a change in weight and a progression towards the patient's target dry weight.

In another aspect, the invention provides a system for characterizing a set of patients undergoing hemodialysis. The system includes: 1) a body-worn biometric sensor, similar to that described above or alternatively having a different configuration (e.g. a patch), that measures ECG, TBI, PPG, and PCG waveforms; 2) a processor for collectively analyzing the ECG, TBI, PPG, and PCG waveforms to determine a set of physiological parameters; 3) a first wireless transceiver configured to transmit the set of physiological parameters; 4) a charging station that receives each body-worn biometric sensor in the set of body-worn biometric sensors; and 5) a gateway system that includes a second wireless transceiver configured to receive the set of physiological parameters from each body-worn biometric sensor in the set of body-worn biometric sensors. The gateway system does this by wirelessly pairing with and then downloading a first set of information from a first body-worn biometric sensor in the set, and then once finished repeating the process with each other sensor in the set. This process is done in a completely automated manner until all information is downloaded. Typically the gateway system then sends it to a web-based system for further review and analysis (e.g. by a data-analytics engine).

In other aspects, the invention provides systems for estimating a fluid level (e.g. TFC) or characterizing BP of a patient undergoing a dialysis session. Here, the systems include sensors, gateway systems, data-analytics engines, and web-based systems similar to those described above.

In still other aspects, the invention provides a sensor for characterizing a patient that includes a set of four electrodes, with two electrodes in the set connected to an electrical circuit configured to inject electrical current into the patient, and two separate electrodes in the set connected to an electrical circuit configured to sense a voltage from the patient's chest. The sensor includes an analog system with a first analog filter that processes the voltage to determine an impedance waveform, and a second analog filter that processes the voltage to determine an ECG waveform. A processor within the sensor processes the ECG waveform to determine a first fiducial point, and the impedance waveform to determine a second fiducial point, and then processes a time difference between the first and second fiducial point to determine a blood pressure value. Here, the sensor is worn completely on the patient's body and also comprising a wireless transmitter for transmitting information to an external gateway system.

Still other advantages should be apparent from the following detailed description, and from the claims.

### BRIEF DESCRIPTION OF THE DRAWINGS

FIG. 1 is a schematic drawing showing a set of patients undergoing hemodialysis, with each patient wearing a sensor that transmits information to a central station coupled to a data-analytics engine according to the invention;

FIG. 2 is a flow chart of an algorithm used with the data-analytics engine of FIG. 1 to predict decompensation occurring in a patient undergoing hemodialysis;

FIG. 3 is a photograph of a front portion of a sensor according to the invention;

FIG. 4A is a photograph of a back portion of the sensor according to the invention, with exposed electrode contact points;

FIG. 4B is a photograph of a back portion of the sensor according to the invention, with disposable patch electrodes connected to the exposed electrode contact points;

FIG. 5A is a time-dependent plot of an ECG waveform collected from a patient;

FIG. 5B is a time-dependent plot of a PCG waveform collected simultaneously and from the same patient as the ECG waveform shown in FIG. 5A;

FIG. 5C is a time-dependent plot of a PPG waveform collected simultaneously and from the same patient as the ECG waveform shown in FIG. 5A;

FIG. 5D is a time-dependent plot of a TBI waveform collected simultaneously and from the same patient as the ECG waveform shown in FIG. 5A;

FIG. 5E is a time-dependent plot of a first derivative of the TBI waveform shown in FIG. 5D;

FIGS. 5A-5E are time-dependent plots of the following waveforms collected from a patient: ECG (FIG. 5A), PCG (FIG. 5B), PPG (FIG. 5C), AC component of TBI (FIG. 5D), and derivative of the AC component of TBI (FIG. 5E);

FIG. 6A is a time-dependent plot of ECG and PPG waveforms generated with the sensor of FIG. 3 and from a single heartbeat from a patient, along with circular symbols marking fiducial points in these waveforms and indicating a time interval related to electro-mechanical activation time (EMAT);

FIG. 6B is a time-dependent plot of TBI and PPG waveforms generated with the sensor of FIG. 3 and from a single heartbeat from a patient, along with circular symbols marking fiducial points in these waveforms and indicating a time interval related to a first VTT (VTT₁);

FIG. 6C is a time-dependent plot of PCG and PPG waveforms generated with the sensor of FIG. 3 and from a single heartbeat from a patient, along with circular symbols marking fiducial points in these waveforms and indicating a time interval related to a third VTT (VTT₃);

FIG. 6D is a time-dependent plot of ECG and PPG waveforms generated with the sensor of FIG. 3 and from a single heartbeat from a patient, along with circular symbols marking fiducial points in these waveforms and indicating a time interval related to PAT;

FIG. 6E is a time-dependent plot of PCG and TBI waveforms generated with the sensor of FIG. 3 and from a single heartbeat from a patient, along with circular symbols marking fiducial points in these waveforms and indicating a time interval related to a second VTT (VTT₂);

FIG. 6F is a time-dependent plot of a PCG waveform generated with the sensor of FIG. 3 and from a single heartbeat from a patient, along with circular symbols marking fiducial points in this waveform and indicating a time interval related to LVET;

FIG. 7A is a schematic drawing of a patient wearing the sensor, whose BP measurement is calibrated using a cuff-based system;

FIG. 7B is a schematic drawing of a patient wearing the sensor after it has been calibrated using a cuff-based system;

FIG. 8A is a photograph of a set of sensors attached to a charging station while information therefrom is wirelessly downloaded;

FIG. 8B is a photograph of a software user interface operating on a tablet computer gateway that downloads information from the set of sensors shown in FIG. 8A;

FIG. 9A is a photograph of a manikin showing where TFC values are measured according to the sensor of the invention (referred to below as a ‘test’ device);

FIG. 9B is a photograph of a manikin showing where Z₀ values are measured with a ‘reference’ device used in a clinical trial described herein;

FIG. 10A is a scatterplot showing impedance values, measured as a function of fluid removed during hemodialysis for both the test and reference devices, where the values for the test and reference devices diverge;

FIG. 10B is a correlation plot showing agreement between measurements made by test and reference devices, as shown in FIG. 10A;

FIG. 10C is a scatterplot showing impedance values, measured as a function of fluid removed during hemodialysis for both the test and reference devices, where the values for the test and reference devices converge;

FIG. 10D is a correlation plot showing agreement between measurements made by test and reference devices, as shown in FIG. 10C;

FIG. 11A is a scatterplot showing impedance values, measured as a function of fluid removed during hemodialysis for both the test and reference devices, where the values for the test and reference devices are relatively high;

FIG. 11B is a correlation plot showing agreement between measurements made by test and reference devices, as shown in FIG. 11A;

FIG. 11C is a scatterplot showing impedance values, measured as a function of fluid removed during hemodialysis for both the test and reference devices, where the values for the test and reference devices are relatively low;

FIG. 11BD is a correlation plot showing agreement between measurements made by test and reference devices, as shown in FIG. 11C;

FIG. 12 is a scatterplot showing the pooled impedance values for a clinical trial conducted with 33 subjects, as measured as a function of fluid removed during hemodialysis for both the test and reference devices;

FIG. 13A is a time-dependent plot of an ECG waveform measured from a patient having a normal sinus rhythm; and

FIG. 13B is a time-dependent plot of an ECG waveform measured from the same patient used to generate the waveform in FIG. 13B, only in this case the patient is experiencing ventricular tachycardia.

### DETAILED DESCRIPTION

### 1. Monitoring ESRD Patients During Hemodialysis

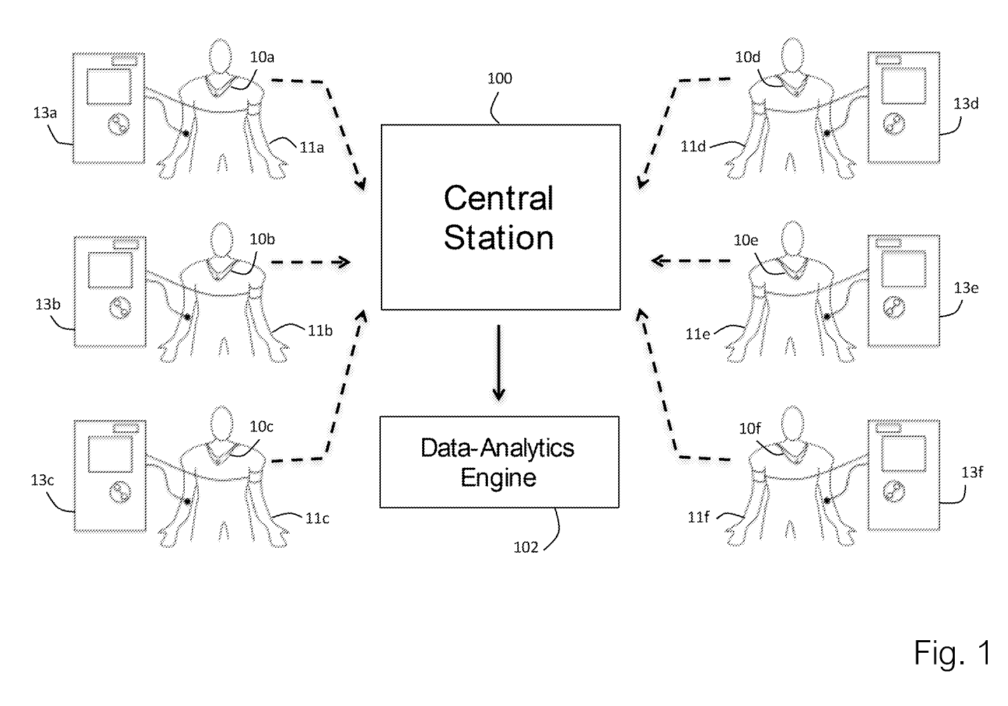

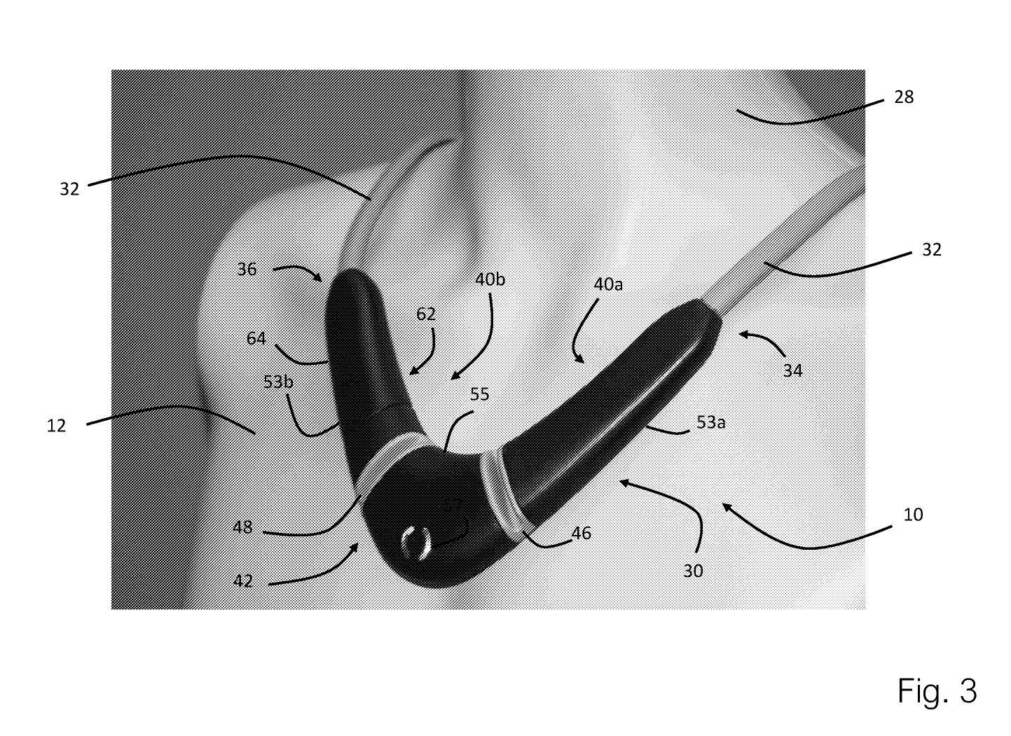

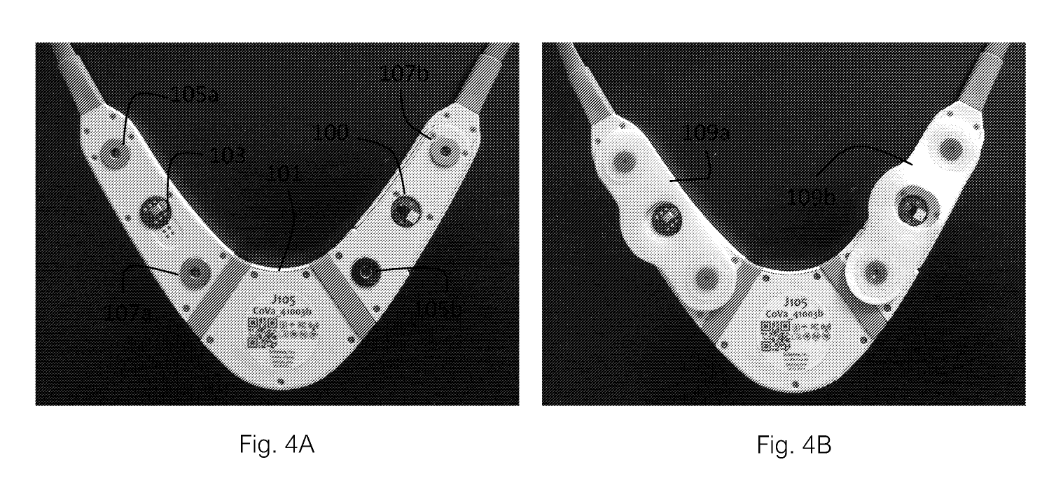

As illustrated in FIG. 1, a sensor 10a-f according to the invention can be used to measure a collection of ESRD patients 11a-f connected to individual dialysis machines 13a-f. The dialysis machines, for example, may be located in a single dialysis clinic. Each sensor 10a-f, as described in more detail below with reference to FIGS. 3 and 4 below, continuously measures a collection of time-dependent physiological waveforms (ECG, TBI, PPG, PCG), vital signs (HR, RR, TEMP, SpO2, and BP) and hemodynamic parameters (TFC, SV, CO) and then wirelessly transmits data indicating these parameters to a central station 100. The sensor 10a-f typically measures waveforms at relatively high frequencies (e.g. 250 Hz) compared to the vital signs and hemodynamic parameters (e.g. once every minute). The sensor 10a-f measures the time-dependent waveforms directly from the patient with embedded sensing elements, described in more detail below. Using computational algorithms, a microprocessor within each sensor 10a-f determines the vital signs and hemodynamic parameters from the time-dependent waveforms. Examples of computational algorithms are described in the following co-pending and issued patents, the contents of which are incorporated herein by reference: “NECK-WORN PHYSIOLOGICAL MONITOR,” U.S. Ser. No. 62/049,279, filed Sep. 11, 2014; “NECKLACE-SHAPED PHYSIOLOGICAL MONITOR,” U.S. Ser. No. 14/184,616, filed Aug. 21, 2014; and “BODY-WORN SENSOR FOR CHARACTERIZING PATIENTS WITH HEART FAILURE,” U.S. Ser. No. 14/145,253, filed Jul. 3, 2014.

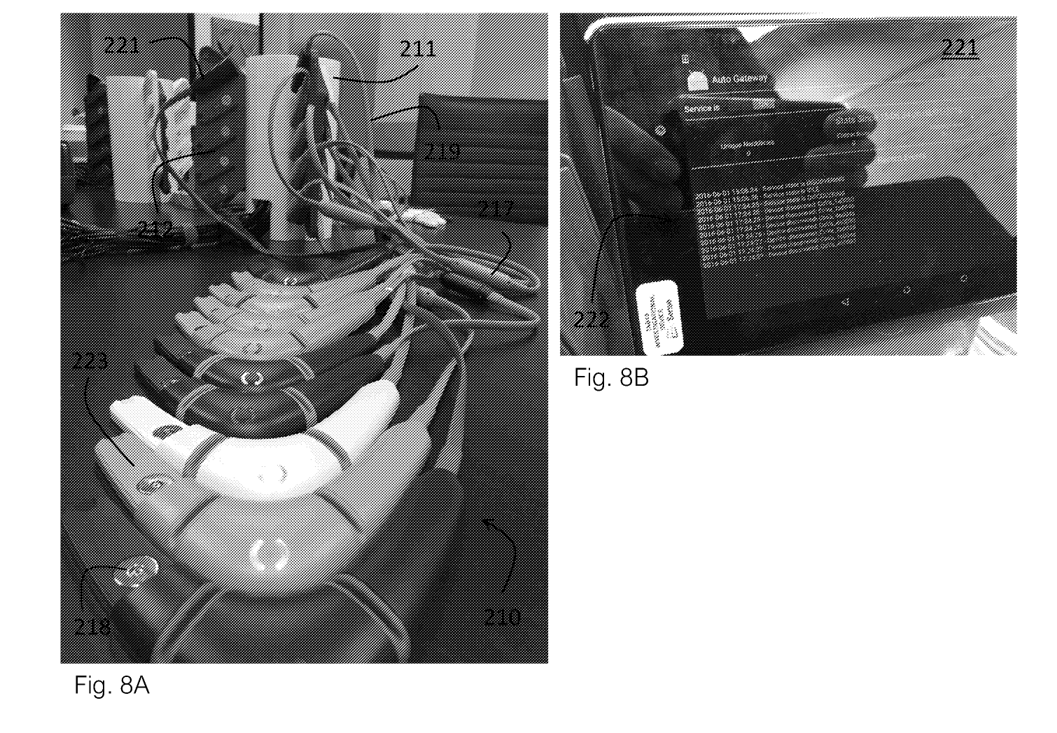

Wireless transmission is typically performed with an internal radio within the sensor 10a-f, such as a radio using protocols based on Bluetooth® or 802.11a-g (referred to herein as WiFi®). The central station 100, for example, can be a computer, workstation, tablet computer, or mobile telephone having a corresponding Bluetooth® or WiFi® radio. Alternatively, each sensor 10a-f wirelessly transmits data to a network operating within the dialysis clinic, and the central station 100 functions as a node on the network to receive the data. In yet another alternate embodiment, described in more detail with reference to FIGS. 8A, 8B, the sensor collects data during a dialysis session, and then stores it in internal memory. The data can then be sent wirelessly (e.g. to the network or central station) at a later time. For example, in the example shown in FIGS. 8A, B, the sensor wirelessly transmits data when its rechargeable battery is being charged (e.g. with a charging station). Here, the gateway is a tablet computer with an internal Bluetooth® transceiver that sequentially and automatically pairs with each sensor attached to the charging station. Once all the data collected during a dialysis session are uploaded to the gateway, the gateway then pairs with another sensor attached to the charging station and repeats the process. This continues until data from each sensor is downloaded.

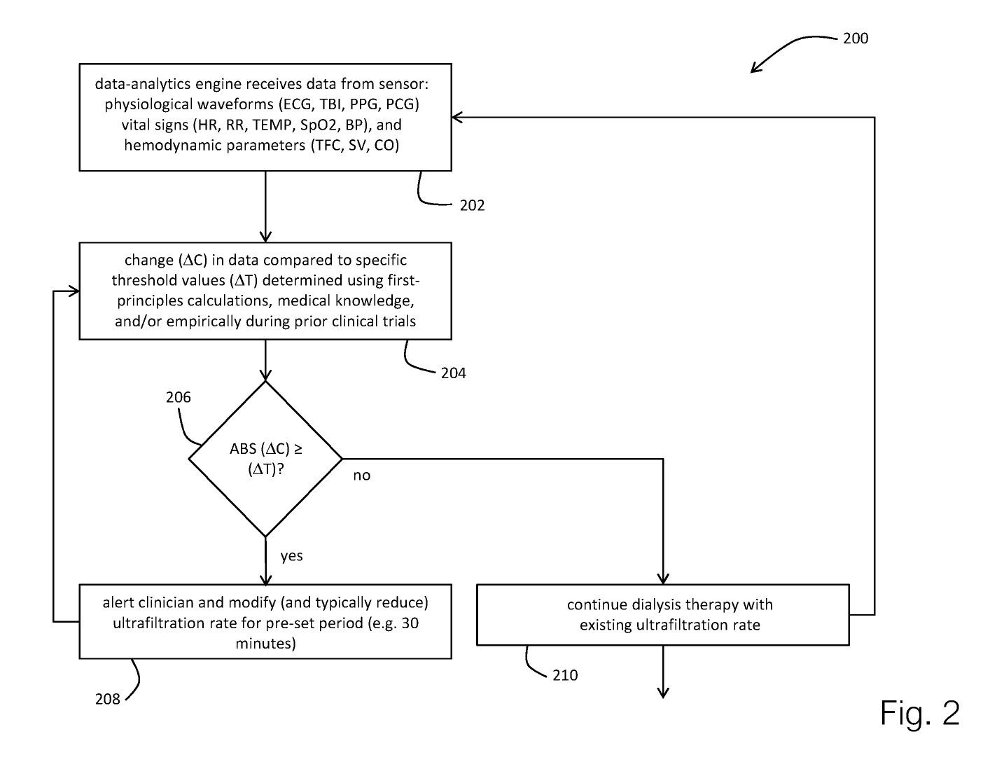

A data-analytics engine 102 in communication with the central station 100 receives and processes the data (time-dependent waveforms, vital signs, and hemodynamic parameters) generated by each patient 11a-f in the dialysis clinic. More specifically, the data-analytics engine 102 is a software system that operates algorithms designed to predict decompensation of the patients 11a-f based on data generated by their respective sensor. Types of decompensation predicted by the data-analytics engine include: 1) rapid changes in vital signs or hemodynamic parameters, e.g. BP, HR, SpO2, RR, TEMP, SV, and CO; 2) hypotension or hypertension; 3) hypoxemia; 4) dysrhythmias; 5) dehydration leading to cramping; 6) chills; 7) nausea; 8) postural changes leading to ineffective therapy; 9) seizures; and 10) rapid blood loss (either internal or external). High-level algorithms for predicting these conditions are described in more detail below with respect to FIG. 2. The goal for these algorithms is to indicate to clinicians working in the dialysis clinic that a particular patient is in the early stages of decompensation, and in response generate an alarm or alert. Clinicians exposed to the alarm or alert may intervene and modify the patient's dialysis therapy to stave off the more severe decompensation before it actually occurs.

FIG. 2 shows a flow chart of an algorithm 200 that may operate on the data-analytics engine 102 shown in FIG. 1 to predict decompensation in a dialysis patient. The algorithm 200 is summarized below:

Step 202: the data-analytics engine receives the following time-dependent data from sensor: physiological waveforms (ECG, TBI, PPG, PCG—sampled every 250 Hz), vital signs (HR, RR, TEMP, SpO2, BP—calculated from the waveforms every 1-15 minutes), and hemodynamic parameters (TFC, SV, CO—calculated every 1-15 minutes). Vital signs and hemodynamic parameters are calculated directly on the sensor using the computational algorithms referenced above.

Step 204: the data-analytics engine calculates changes (ΔC) in one or more of the data from step 202, and compares values of ΔC to specific threshold values (ΔT) determined empirically using first-principles calculations, medical knowledge, and/or prior clinical trials. Typically the values of ΔT correspond to significant changes that lead to conditions such as: 1) rapid changes in BP leading to hypotension and hypertension; 2) hypoxemia; 3) dysrhythmias; 4) dehydration leading to cramping; 5) chills; 6) nausea; 7) postural changes leading to ineffective therapy; 8) seizures; and 9) rapid blood loss (either internal or external).

Step 206: compare ΔC to ΔT for one or more parameters collected during step 204, and determine in each case if ΔC exceeds ΔT.

Step 208: if ΔC exceeds ΔT for one or more parameter, alert clinician using an alarm (e.g. audio, visual alarm); at this point the clinician may modify the patient's ultrafiltration rate.

Step 210: if ΔC does not exceed ΔT for any parameter, continue dialysis therapy with the existing ultrafiltration rate.

Table 1 below describes examples of ΔT values for each of the vital signs and hemodynamic parameters measured by the sensor.

TABLE 1
Physiological parameters measured from patients undergoing
hemodialysis and corresponding values of ΔT.

| Parameter | ΔT | |
|---|---|---|
| HR | 20 | beats/min |
| RR | 5 | breaths/min |
| TEMP | 3° | F. |
| SpO2 | 5% | |
| BP | 20 | mmHg |
| TFC | 5 | Ohms |
| SV | 20 | mF |
| CO | 2 | L/min. |

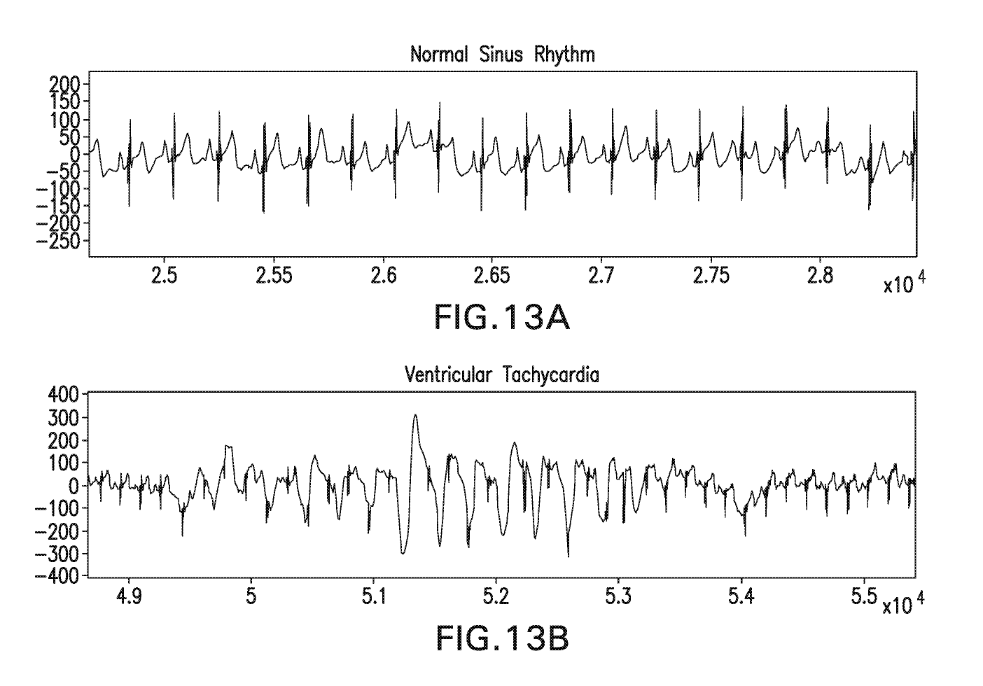

In other embodiments, specific properties of the time-dependent waveforms may be processed by the data-analytics engine, which in response may trigger an alarm or alert. For example, as shown by the waveforms in FIGS. 13A,B, during dialysis the ECG waveform measured by the sensor may indicate a change from a normal sinus rhythm (FIG. 13A) to a state of ventricular tachycardia (FIG. 13B). In less severe cases, a simple change in the amplitude of a set of heartbeat-induced pulses, or a component of an individual pulse (e.g. a heartbeat-induced pulse, or a derivative thereof), within the waveform may trigger an alarm or alert. In still other embodiments, the above-described component in the waveform may be correlated to another parameter (e.g. a physiological parameter), a change in which may trigger the alarm.

### 2. Sensor

The sensor described herein, along with its measurements of ECG, TBI, PPG, and PCG waveforms, and its determination of HR, HRV, RR, SpO2, BP, TFC, SV, and CO values from these waveforms, is described in detail in the above-described co-pending patent applications, the contents of which have been previously incorporated herein by reference.

As illustrated in FIG. 3, a sensor 10 according to the invention is designed to monitor a patient during hemodialysis. As indicated above and explained in greater detail below, the sensor 10 measures numerical and waveform data, and then sends this information wirelessly to a central station and data-analytics engine within the dialysis clinic. The sensor 10 is typically worn around the patient's neck 28 so that it rests against their sternum, similar to a necklace or other neck-adorning jewelry. The sensor 10 features a sensing portion 30 and a securement member 32 (or securement members in an alternate embodiment, not illustrated). As illustrated, the securement member 32 extends from a first end 34 of the sensing portion 30 and attaches to a second end 36 of the sensing portion 30. The securement member 32 is long enough to pass behind the patient's neck 28 and to hold the sensing portion 30 in proper position for sensing electrodes attached to its rear, patient-facing surface to be attached to the proper locations on the patient's chest. This ensures that the sensing portion 30 is placed in approximately the same position for each measurement made on a particular patient, and that it is held in proper position to acquire the relevant bioelectric signals, as explained more fully below. Additionally, the securement member 32 houses a battery in battery compartment 38, which is positioned generally in the middle of the securement member 32 (lengthwise speaking) such that it is positioned inconspicuously behind the patient's neck 28 when the sensor 10 is worn.

In other, non-illustrated embodiments, the securement member could be split in the middle, with flexible yet shape-retaining “branches” extending from the first and second ends 34, 36 of the sensing portion 30 so as to pass behind the patient's neck 28, but not connect, much like a physician's stethoscope. In that case, the battery compartment could be located in one of the branches or, alternatively, in the sensing portion 30 of the sensor 10. In still further non-illustrated embodiments, a securement member might not be included, in which case attachment of the electrodes to the patient's body would, by itself, be used to hold the sensor in position.

In still other embodiments, the sensor 10 may lack the securement member 32 and only include the sensing portion 30. In this case, the system has an internal battery and resembles a ‘patch’ instead of the necklace shown in FIG. 3. The patch (and corresponding sensing portion) can feature several different geometries. For example, it may be shaped like a large Band-Aid®, or have an elongated ‘racetrack’ geometry. The patch may be work near the center of the patient's chest, as shown in FIG. 3, or on the left or right-hand side of the chest.

The sensing portion 30 is typically constructed in two or more sections or segments, e.g. a central segment 42 and two outboard segments 40a and 40b. Electrode patches attach to the rear of the two outboard segments 40a and 40b, as described below. The segments are connected to each other by means of flexible connector segments (not shown in the figure), which in turn are encased in flexible housing 46 and 48. The flexible connector segments are typically made from a polymeric material, e.g. Kapton® flexible printed circuits available from the DuPont Corporation. Such materials are essentially a flexible, polymeric film that encases one or more thin conducting members, which are typically made from copper. Each of the segments 40a, 40b, and 42 includes, respectively, a rigid circuit board (not shown in the figure) populated with discrete electrical circuit components, described in more detail below. The rigid circuit boards connect to one another via the flexible connector segments, which each include 20 conductive members.

The rigid circuit boards are each encased inside of a rigid protective housing segments 53a, 53b, 55, and the flexible connector segments are encased within the flexible connector segments 46 and 48. The protective housing segments 53a, 53b, and 55 are more typically made from opaque plastic, which contributes to the overall aesthetically pleasing appearance of the sensor 10. Suitably, the connector segments 46 and 48, which may be formed as rubber boots designed to snap into respectively opposing ends of the protective housing segments 53a, 53b, 55, are typically made from soft, flexible material such as silicone rubber. Generally speaking, such a configuration of the sensing portion 30 serves to hold the sensing electrodes at their proper positions before they are adhered to the patient's chest, while allowing the sensing portion 30 to conform to the different curvatures of the physiological region upon which it rests.

A transparent or translucent plastic window 57 located on the top, anteriorly facing surface of central housing segment 55 covers an underlying LED, which serves as a simple user interface for the patient 12. For example, the LED can radiate different colors of the visible spectrum, and blink them at different frequencies, to indicate when the sensor 10 is turned on, making a measurement, charging, running on low power, completed with a measurement, etc. Additionally included in the sensor are an acoustic ‘buzzer’ and/or vibrating component. Collectively, the LED, buzzer, and vibrating component can alert the clinician in case of an alarm, triggered as described above.

As shown in FIGS. 4A and 4B, on its rear-facing surface 101 the sensor 10 includes a pulse oximetry sensor 100 that operates using reflection-mode optics, and an acoustic sensor 103 featuring a piezoelectric microphone that measures sounds generated when valves close in the patient's heart. The pulse oximetry sensor 100 and acoustic sensor 103 generate, respectively, PPG and PCG waveforms during a measurement, as described in more detail below. Such waveforms can be further processed to determine SpO2, BP, SV, CO and other parameters. The pulse oximetry sensor 100 and acoustic sensor 103 are disposed on the back surface of opposing housing segments (53a and 53b in FIG. 3), between magnetic interfaces for sense 105a,b and drive 107a,b electrodes associated with the ECG and impedance circuits. During a measurement, as indicated in FIG. 4B, stainless steel posts embedded within two separate electrode patches 109a, 109b connect to the magnetic interfaces for sense 105a,b and drive 107a,b electrodes. Each electrode patch 109a, 109b includes an aperture (i.e. a cut-out circular hole) so that the underlying sensing element (pulse oximetry sensor 100 and acoustic sensor 103) can directly contact the patient's skin when the sensor is worn. Measurements are then made as described in more detail below.

The above-described patent applications, which have been incorporated herein by reference, describe how the sense and drive electrodes measure both ECG and TBI waveforms. To summarize, the drive electrodes inject high-frequency, low-amperage current into the patient's chest. The sense electrodes sense a voltage that indicates the impedance encountered by the injected current. The voltage is passed through a series of electrical circuits featuring analog filters and differential amplifiers to filter out and amplify signal components related to the two different waveforms. This is done using techniques known in the art, and described in the patent applications. One of the signal components indicates the ECG waveform. Another indicates the TBI waveform. The TBI waveform has low-frequency and high-frequency components that are further filtered out and processed, as described in more detail below, do determine different impedance waveforms.

An example of a pulse oximetry sensor is described in U.S. Pat. No. 8,437,824, the contents of which are incorporated by reference in their entirety. The pulse oximetry sensor 100 drives red and infrared LEDs in an alternating, pulsatile manner and controls a light-sensitive, photodetector diode, as generally known in the art. It is configured to operate in a reflection mode, meaning that the LEDs and light-sensitive diode are positioned so as to receive radiation from the same direction. It measures PPG waveforms from capillary beds in the patient's chest to generate a value of SpO2. This is in contrast to conventional pulse oximetry sensors in which the LEDs and the light-sensitive diode are positioned across from each other, with a space into which fits a body part (e.g., a finger or an earlobe) being located between the LEDs and the light-sensitive diode. Thus, the pulse oximetry circuit detects and measures radiation emitted by the diodes that has been reflected off of capillary beds (i.e., in the chest) before arriving at the light-sensitive diode.

The acoustic sensor 103 typically includes a microphone (e.g. a piezoelectric microphone) and amplifier system, and is designed to detect a PCG waveform indicating heart sounds, primarily caused by the closings of the atrioventricular and semilunar valves during each heartbeat. Alternatively, a sensitive accelerometer can be used in place of the acoustic sensor 103 to measure small-scale, seismic motions of the chest driven by the patient's underlying beating heart. Such waveforms are referred to as seismocardiogram (SCG) and can be used in place of (or in concert with) PCG waveforms.

Because both the pulse oximetry sensor 100 and acoustic sensor 103 are incorporated into the overall sensor 10, they can connect comfortably to the patient's chest to measure signal in an effective manner that eliminates “cable clutter” and frees the patient's hands and fingers (where pulse oximetry measurements typically are taken) for other purposes. An additional benefit of this configuration is reduction of motion artifacts, which can distort PPG waveforms and cause erroneous values of SpO2 to be reported. This reduction of motion artifacts is due to the fact that during everyday activities, the chest typically moves less than the hands and fingers, and subsequent artifact reduction ultimately improves the accuracy of parameters measured from the patient.

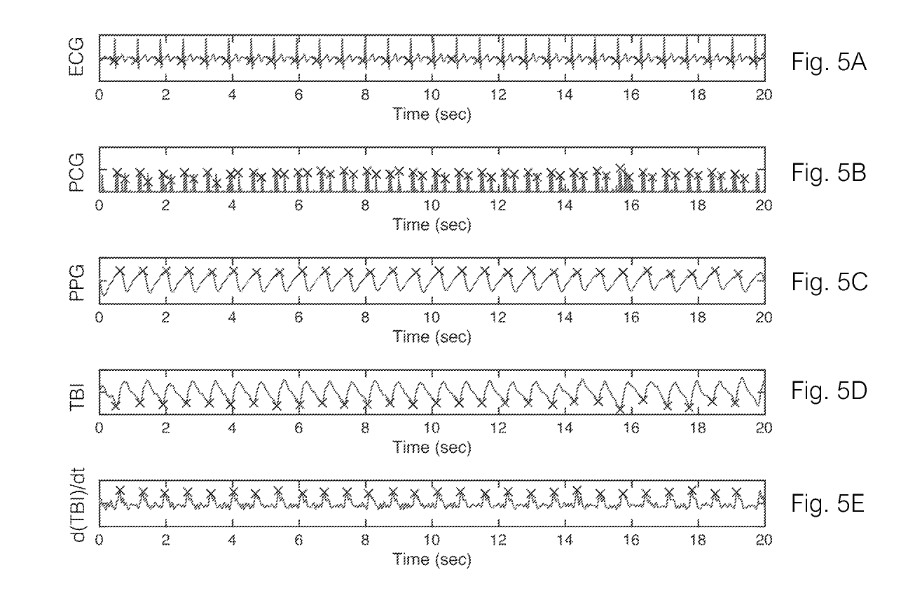

FIGS. 5A-E shows time-dependent plots of ECG, TBI, PPG, and PCG waveforms measured by the sensor according to the invention, along with ‘x’ symbols indicating fiducial points in the waveforms determined by feature-detecting firmware operating on the sensor. As described in detail below, the sensor measures a collection of physiological signals by collectively processing all four waveforms.

BP, including SYS and DIA, is particularly relevant for ESRD patients, as they can easily enter into hypertensive and (more commonly) hypotensive states during hemodialysis treatments. Measurement of BP with the sensor is therefore discussed in more detail here. The sensor monitors BP by simultaneously tracking the physiologic waveforms shown in FIGS. 5A-E. The ECG waveform shown in FIG. 5A includes a heartbeat-induced QRS complex that informally marks the beginning of each cardiac cycle. Following this is a PCG waveform—captured with the acoustic sensor and shown in FIG. 5B—indicates heart sounds. Following this is a PPG waveform—captured with the pulse oximetry sensor and shown in FIG. 5C—that monitors volumetric changes in underlying capillaries. The TBI waveform includes DC (Z₀) and AC (ΔZ) components: Z₀ senses the amount of fluid in the chest by measuring underlying electrical impedance and represents the baseline of the waveform; AZ tracks blood flow in the thoracic vasculature and represents the pulsatile components of the waveform (as shown in FIG. 5D).

The QRS complex provides a fiducial marker to delineate each heartbeat. Feature-detection algorithms operating in the sensor calculate time intervals between the QRS complex and fiducial markers on each of the other waveforms. For example, the time separating a ‘foot’ of a pulse in the PPG waveform and the QRS complex is referred to as PAT. PAT relates to BP and systemic vascular resistance. During a measurement, the sensor calculates PAT, along with VTT and other time-dependent parameters extracted from the four physiologic waveforms (collectively referred to below as ‘INT’). Additionally, the sensor calculates information about the amplitudes of heartbeat-induced pulses in some of the waveforms (‘AMP’). For example, the amplitude of the pulse in the derivative of the AC component of the TBI waveform (dZ/dtₘₐₓ), as shown in FIG. 5E, indicates the volumetric expansion and forward blood flow of the thoracic arteries, and is related to SYS and the contractility of the heart.

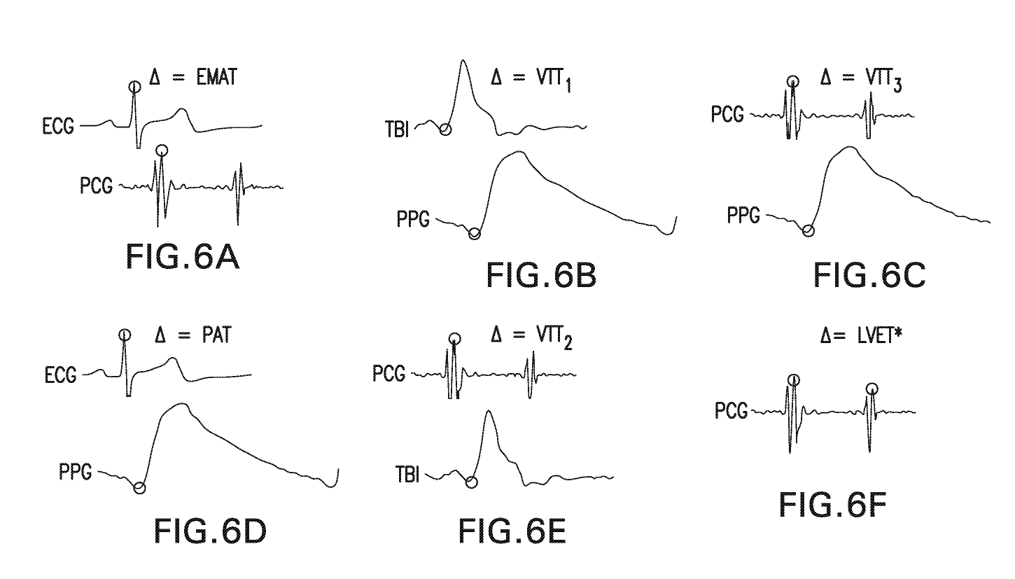

The general model for calculating SYS and DIA involves measuring a collection of INT and AMP values from the four physiologic waveforms. FIGS. 6A-6E, for example, show a collection of INT values that may correlate to BP. These include: 1) EMAT, shown in FIG. 6A, which is the time separating an ECG QRS and the onset of the S1 heart sound; 2) VTT₁ (FIG. 6B) which is the time separating the onset of pulses in the TBI and PPG waveforms; 3) VTT₃ (FIG. 6C) which is the time separating the onset of the S1 heart sound and the onset of a pulse in the PPG waveform; 4) PAT (FIG. 6D) which is the time separating the ECG QRS and the onset of a pulse in the PPG waveform; 5) VTT₂ (FIG. 6E) which is the time separating the onset of the S1 heart sound and the onset of a pulse in the TBI waveform; and 6) LVET (FIG. 6F) which is the time separating the S1 and S2 heart sounds. Note from Eqn. 3 above, LVET is typically estimated directly from a cardiac pulse in the TBI waveform, or from the patient's current HR using Weissler's regression. Errors in these estimations may lead to errors in calculating SV. Determining LVET directly from S1 and S2 may reduce such errors, and thus improve the accuracy of the calculated SV.

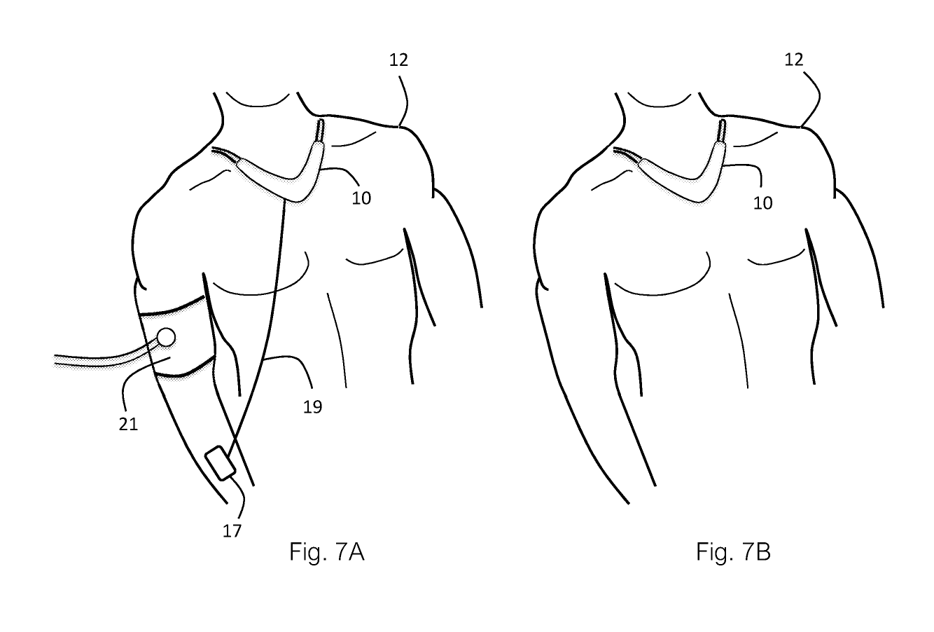

Once these parameters are determined, firmware on the sensor then collectively processes them, along with demographic information (e.g. age and gender) and information measured during a patient-specific calibration described below with reference to FIGS. 7A and 7B, to determine BP values without requiring a cuff. Eq. 5 below, for example, shows one example of an algorithm (e.g. an equation) for determining BP from the parameters shown in FIGS. 6A-F. In the equation, coefficients a-f are determined during the calibration.

BP=a×EMAT+b×VTT₁+c×VTT₃+d×PAT+e×VTT₂+f×LVET (5)

This allows, for example, BP values (SYS and DIA) to be monitored in a quasi-continuous manner (e.g. every minute or so) during hemodialysis, thereby allowing detection of rapid excursions into hypertensive and hypotensive states.

The sensor according to the invention also typically includes a three-axis digital accelerometer and a temperature sensor (not specifically identified) to measure, respectively, three time-dependent motion waveforms (along x, y, and z-axes) and TEMP values.

### 3. Measurement of Stroke Volume

According to the invention, algorithms for calculating SV and other physiological parameters (e.g. BP, SpO2, RR, CO) are described in more detail in the co-pending patent applications described above, the contents of which have been previously incorporated herein by reference. The algorithms described therein can be improved upon by collective processing of time-dependent TBI and PCG waveforms, as indicated in FIG. 6. The figure shows a plot of ‘pulses’ induced by a single heartbeat for these waveforms. To measure SV, a microprocessor within the sensor first processes the PCG waveform, and specifically the temporal delay between the S1 and S2 heart sounds therein (as indicated in FIG. 6F), to determine LVET, which is then used in various forms of the SV equation, as described in the above-referenced patent applications, to determine SV. Once determined, SV is further processed to determine PP, which is then further processed to determine SYS and DIA. The invention also includes secondary algorithms for converting SV into CO, and processing S1 and S2 to determine a patient's cardiac function.

In alternative embodiments, the invention may include use a signal-processing technique called ‘beatstacking’ to improve the signal-to-noise ratio of heartbeat-induced pulses in the TBI waveform. With beatstacking, an average pulse—Z(t)—is calculated from multiple (e.g. seven) consecutive pulses from the TBI waveform, which are delineated by an analysis of the corresponding QRS complexes in the ECG waveform, and then averaged together. The derivative of Z(t)—dZ(t)/dt—is calculated over an 8-sample window. The maximum value of Z(t) is calculated, and used as a boundary point for the location of [dZ(t)/dt]ₘₐₓ. This parameter is used directly in the SV equation, described above.

### 4. Calibrating the Blood Pressure Measurement

Measurement of BP made by the sensor during dialysis must be calibrated with a cuff-based system. A preferred approach, illustrated in FIGS. 7A and 7B, uses a ‘BP calibration device’ that features a cuff-based oscillometric measurement. The BP calibration device is typically included directly in the machine used for hemodialysis. Alternatively it can be included in an off-the-shelf device separate from the dialysis machine. Calibration is typically performed at the start of each dialysis session. As shown in FIGS. 7A and 7B, it requires a sensor 10 disposed on the chest of a patient 12, and a third disposable patch electrode 17, identical to the 2-part electrodes that attach directly to the sensor's base and shown in FIG. 4B, which adheres to a patient's wrist or forearm. During a calibration, the third electrode 17 connects through a thin cable 19 (about 3 feet in length) to the sensor's base. A BP cuff 21 associated with the BP calibration device is placed on the same arm as the third electrode.

Calibration begins when the user (either a patient or clinician) presses a button labeled ‘Calibration’ on a user interface of the tablet computer gateway (not shown in the figure). The user interface asks for input of certain biometric parameters corresponding to the patient (e.g. age, gender), and then prompts the user to initiate an oscillometric BP measurement with the BP calibration device. This process establishes a Bluetooth® connection between the gateway and the BP calibration device. The Sensor then begins to measure PCG and PPG waveforms from the patient's chest, and ICG and ECG waveforms between the third electrode on the patient's wrist/forearm and one of the electrodes adhered to their chest. Over Bluetooth®, the BP calibration device transmits DC (‘PRES-DC’) and AC (‘PRES-AC’) pressure waveforms to the sensor. These represent, respectively, the background pressure that the cuff 21 applies to the patient's brachial artery during the measurement and the oscillometric envelope. Algorithms within the sensor 10 synchronize its four waveforms with the PRES-DC and PRES-AC waveforms measured by the BP calibration device. Upon completion of the oscillometry measurement, the BP calibration device also transmits initial BP values (SYS₀, DIA₀, and MAP₀) to the sensor.

Firmware within the sensor then collectively processes the waveforms with a computational model to determine the first component of the calibration: a patient-specific relationship between INT, AMP and changes in BP. These are indicated by coefficients a-f, shown above in Eq. 5. The second component of the calibration is SYS₀, DIA₀, and MAP₀. Collectively these two components represent a calibration, which holds for the entire dialysis session. Once the sensor calculates the calibration, it notifies the gateway over Bluetooth®, which prompts the patient to remove both the third electrode and the BP cuff. Cuffless measurements of BP can then commence with the sensor.

5. Charging Sensors and Downloading Information after a Dialysis Session

FIGS. 8A and 8B show a collection of sensors 210 attached to a charging station 211. The charging station, for example, may be used in a dialysis clinic to charge the sensor between dialysis sessions. Each sensor 210 is powered by a rechargeable Li:ion battery 217, which as described above is located in its securement member or ‘cable’ 219. During a measurement, the cable 219 loops around the neck of a patient so that the battery 217 is tucked behind their neck. The sensor 210 is powered on when its clasp 221 snaps into a mated magnetic interface 218 on the sensor's base 223. This action completes a power circuit within the sensor, causing it to power on. Measurements then commence. Before or after a dialysis session, a large number of sensors 210 may require charging of their Li:ion batteries 217. A charging station 211 including multiple ports 212 connects to the sensors 210 to charge their batteries 217. The charging station 211 is plugged into a mains outlet through a plug. Each port 212 on the charging station 211 includes a magnet and a plastic component designed to mate with the clasp 221 (and magnetic interface 218) of each sensor 210. When the clasp 221 is connected to the charging station 211, wires in the cable 219 connect each sensor's battery 217 to a corresponding port 212. Power from the mains outlet then charges the battery 217.

As indicated in FIG. 8B, during the charging process data collected from a set of patients undergoing dialysis can be automatically downloaded from each sensor, and then forwarded to a central gateway or cloud-based system for follow-on analysis. For example, during charging, a tablet computer gateway 221 can be placed proximal to the charging station so that its internal Bluetooth® transceiver is within range of corresponding Bluetooth® transceivers within each sensor. The tablet computer gateway 221 can run a software program featuring a customized user interface 222 that automatically locates each Bluetooth® transceiver within each sensor, pairs with it, and the downloads the data collected during dialysis and stored on internal memory within the sensor. Once data is collected from one sensor, the tablet computer gateway ‘finds’ a subsequent sensor, and repeats the downloading process. This continues until data are downloaded from each sensor. The tablet computer gateway 221 then forwards the downloaded data to a secondary computer system, e.g. a web-based computer system, for follow-on analysis.

### 6. Clinical Results

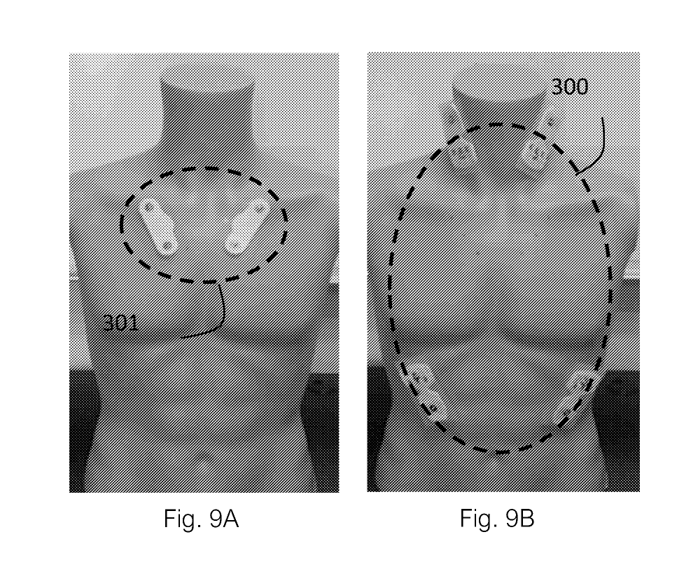

A clinical study with patients undergoing hemodialysis was performed using the sensor described herein, and clearly demonstrates its ability to measure some of the above-described parameters, and particularly TFC. During the study, the sensor (referred to below as the ‘test’ device) measured TFC as described above, and measurements of Z₀ (a parameter related to the inverse of TFC) were made with a second ‘reference’ device, the Cardiodynamics BioZ. FIGS. 9A and 9B show the electrode positions for these two devices. Circle 300 in FIG. 9B shows the BioZ's electrode position, indicating its measurement of Z₀ represents an impedance value for the entire thoracic cavity. Physiological components such as blood, bone, and thoracic fluids, contribute to its value. In contrast, the test device's measurement of TFC, shown by circle 301 in FIG. 9A, represents an impedance value from a localized and relatively small region near the sternum. For this reason, when deployed on a patient, the test device's measurement of TFC and its associated sensitivity to fluid changes are expected to have lower values than those from corresponding measurements made by the reference device. All measurements were made on patients undergoing hemodialysis.

The test device uses only four electrodes, as compared to eight for the reference device. Two electrodes in the test device inject current for the bioimpedance measurement, compared to four electrodes for the reference device. Both the test and reference devices inject a high-frequency, low-amperage current: for the test device the frequency is 100 KHz and amperage is about 6 mA, compared to about 70 KHz and 4 mA for the reference device. Like the reference device, the test device measures both AC and DC waveforms, with its TFC value representing a 30-second average of the DC waveform.

Table 2 summarizes data collected from each subject in the first cohort, and includes: 1) BIAS and STDEV between test and reference device; 2) correlation between measurements made by test and reference devices; 3) correlation between measurements made by test/reference devices and the amount of fluid removed during dialysis; and 4) sensitivity (i.e. a slope with units of Ohms/L) of measurements made by both test and reference devices. The last row in the table shows an average of all these values. Here, the sensitivity accounts for intravenous saline disposed into the subject during dialysis; this value was typically 500 mL.

TABLE 2
Summary of statistics for the clinical study performed with test and referenced devices.

| | | | r - | | r - | | |
|---|---|---|---|---|---|---|---|
| | BIAS | STDEV | correlation | r - | correlation | | |
| | (test device, | (test device, | (test device, | correlation | (reference | | sensitivity |
| | reference | reference | reference | (test device, | device | sensitivity | (reference |
| | device, | device, | device, | fluid removed, | fluid removed, | (test device, | device, |
| Subject | units Ohms) | units Ohms) | no units) | no units) | no units) | units Ohms/L) | units Ohms/L) |
| 300 | 4.56 | 0.88 | 0.62 | 0.82 | 0.88 | 1.08 | 1.08 |
| 301 | 14.07 | 0.83 | 0.93 | 0.98 | 0.97 | 1.38 | 1.95 |
| 303 | 8.30 | 1.04 | 0.89 | 0.94 | 0.97 | 1.92 | 2.64 |
| 304 | 5.33 | 1.39 | 0.84 | 0.80 | 0.96 | 2.40 | 1.80 |
| 306 | 21.35 | 2.70 | 0.68 | 0.88 | 0.90 | 1.03 | 2.51 |
| 307 | 6.93 | 0.82 | 0.92 | 0.91 | 0.97 | 0.48 | 1.56 |
| 309 | 6.81 | 0.72 | 0.87 | 0.90 | 0.87 | 0.68 | 1.02 |
| 310 | 16.95 | 1.03 | 0.89 | 0.94 | 0.91 | 1.46 | 2.43 |
| 311 | 10.78 | 0.99 | 0.95 | 0.96 | 0.93 | 0.77 | 1.33 |
| 314 | 8.97 | 1.62 | 0.87 | 0.88 | 0.99 | 2.12 | 3.41 |
| 316 | 2.63 | 0.67 | 0.66 | 0.95 | 0.84 | 1.54 | 1.02 |
| 318 | 5.38 | 0.50 | 0.97 | 0.98 | 0.99 | 3.10 | 3.10 |
| 319 | 11.95 | 1.39 | 0.89 | 0.98 | 0.90 | 1.92 | 3.00 |
| 321 | 5.76 | 0.99 | 0.67 | 0.96 | 0.82 | 2.20 | 1.68 |
| 322 | 5.79 | 1.37 | 0.97 | 0.99 | 0.97 | 3.16 | 2.20 |
| 323 | 3.65 | 0.61 | 0.94 | 0.94 | 0.99 | 3.27 | 3.27 |
| 324 | 10.02 | 0.91 | 0.88 | 0.91 | 0.95 | 2.18 | 2.05 |
| 326 | 15.18 | 0.45 | 0.94 | 0.95 | 0.95 | 1.39 | 1.90 |
| 329 | 12.12 | 1.11 | 0.90 | 0.97 | 0.95 | 2.62 | 2.02 |
| 331 | 10.87 | 1.24 | 0.93 | 0.93 | 0.99 | 1.76 | 2.21 |
| 332 | 7.75 | 0.72 | 0.92 | 0.98 | 0.89 | 4.34 | 2.89 |
| 335 | 3.73 | 0.38 | 0.73 | 0.54 | 0.81 | 1.13 | 1.46 |
| 337 | 4.98 | 0.80 | 0.75 | 0.84 | 0.94 | 1.30 | 1.18 |
| 338 | 5.12 | 0.72 | 0.96 | 0.95 | 0.99 | 0.57 | 1.15 |
| 339 | 12.44 | 0.94 | 0.93 | 0.94 | 0.99 | 0.88 | 1.43 |
| 340 | 4.02 | 0.85 | 0.91 | 0.96 | 0.96 | 1.50 | 1.04 |
| 342 | 14.61 | 1.25 | 0.88 | 0.95 | 0.89 | 1.85 | 4.26 |
| 343 | 9.09 | 1.07 | 0.84 | 0.98 | 0.91 | 1.00 | 1.23 |
| 344 | 8.70 | 0.99 | 0.88 | 0.88 | 0.95 | 0.82 | 1.75 |
| 345 | 16.71 | 1.27 | 0.88 | 0.84 | 0.94 | 1.26 | 2.06 |
| 346 | 7.84 | 1.86 | 0.77 | 0.91 | 0.93 | 2.87 | 5.08 |
| 348 | 16.32 | 0.94 | 0.91 | 0.92 | 0.99 | 2.08 | 2.93 |
| 349 | 4.56 | 0.61 | 0.69 | 0.88 | 0.92 | 0.88 | 1.25 |
| AVE | 9.19 | 1.02 | 0.86 | 0.91 | 0.93 | 1.72 | 2.12 |

From the data shown in Table 2, it is noted that the average sensitivities for both test (1.68 Ohms/L) and reference (2.02 Ohms/L) devices are similar to those calculated with a mixed-effects statistical model (1.69 Ohms/L and 1.88 Ohms/L, respectively).

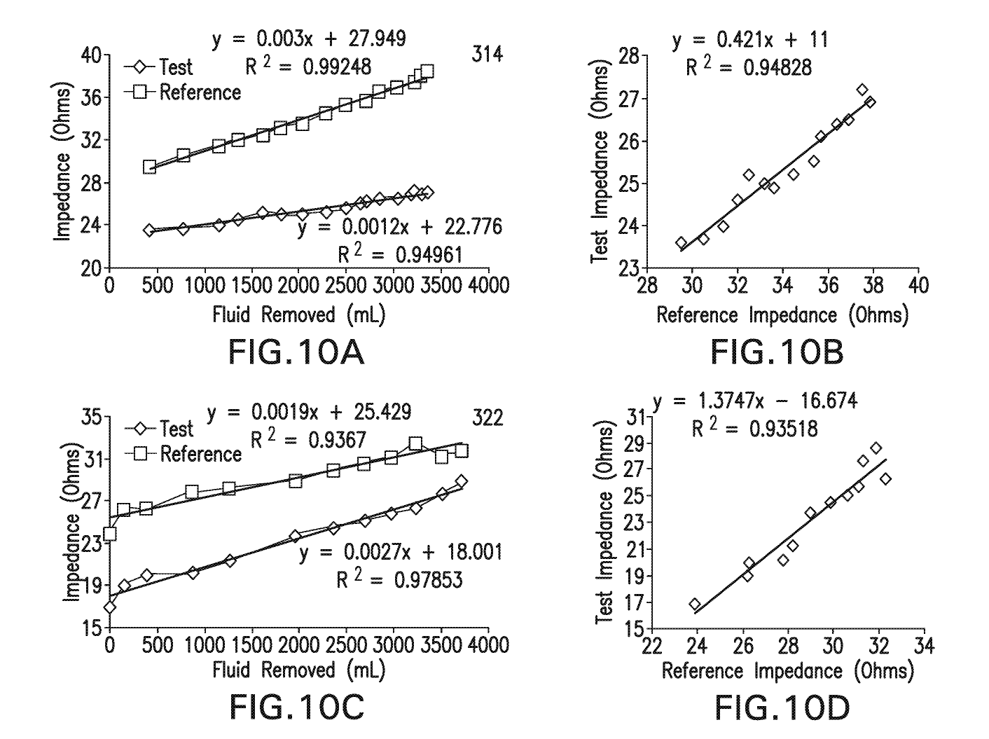

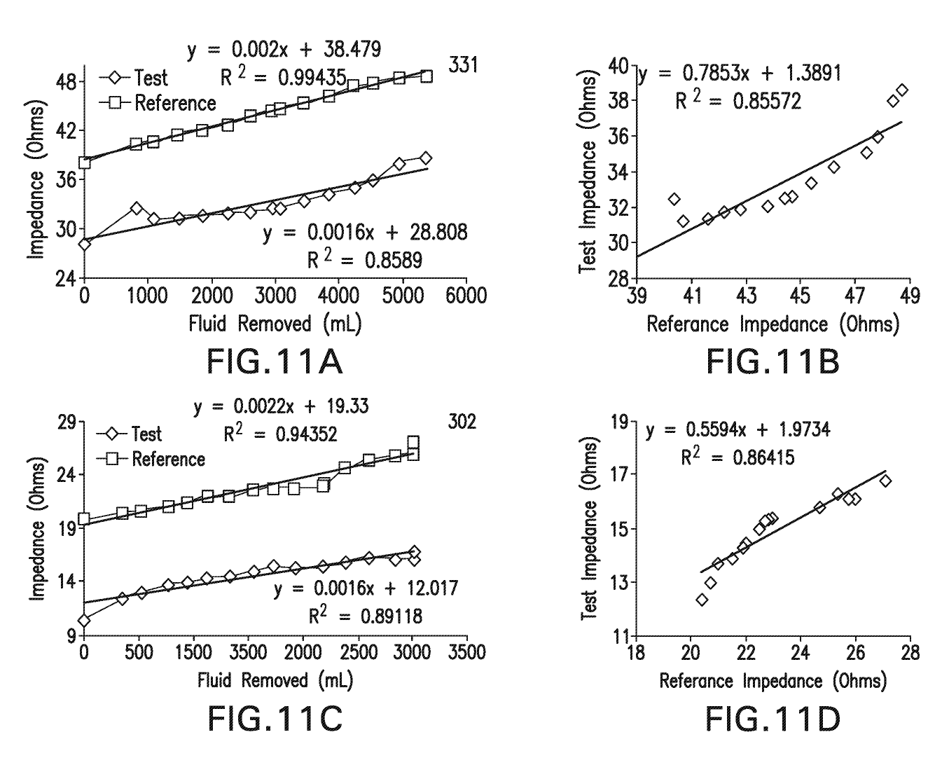

FIGS. 10A-D, 11A-D, below, show plots from 4 subjects (314, 322, 331, 302) indicating the dependence of impedance measurements made by the test and reference devices with fluid removed during dialysis (FIGS. 10A, 10C, 11A, 11C). Corresponding plots (FIGS. 10B, 10D, 11B, 11D) are standard correlation plots showing the agreement between measurements made by the two devices.

Data from the four subjects described above is shown because of the disparate way their impedance values relate to fluid removed. For example, impedance values for subject 314 (FIGS. 10A,B) diverge with increased amounts of fluid removed, while those from subject 322 (FIGS. 10C,D) converge. Likewise, impedance values for subject 331 (FIGS. 11A,B) are some of the highest values recorded, while those for subject 302 (FIGS. 11C,D) are some of the lowest. In all cases, the fluid-dependent plots indicate a strong linear relationship between the impedance values from both test and reference devices and the amount of fluid removed. The correlation plots indicate there is also strong linear relationship between values measured by the two devices.

Importantly, these data clearly appear to show that bioimpedance measurements made by the test device from a relatively small region on the sternum (e.g. circle 301 in FIG. 9A) are sensitive to a patient's fluid variations throughout their thoracic cavity (e.g. circle 300 in FIG. 9A). As expected, bioimpedance values measured from this region have a lower overall value (average BIAS of 9.16 Ohms) and sensitivity (average 1.69 Ohms/L) to fluid changes compared to those measured from the entire thoracic cavity (average 1.88 Ohms/L). This is because measurements from the sternum sample a relatively small physiological area with a correspondingly low fluid volume. However, as shown in Table 2, during periods of fluid removal a strong linear relationship was observed between test and reference devices in 100% of the subjects. Likewise, in 100% of these subjects, measurements made by the test device showed a strong linear relationship and sensitivities to the amount of fluid removed during dialysis.

For the study described above, averaged values measured from subjects with ESRD alone (i.e. cohort 1A with 23 total subjects) were compared to those with both ESRD and CHF (cohort 1B with 10 total). Table 3, below, summarizes these results.

TABLE 3
Averaged statistical values for cohort 1A and 1B.

| | | | AVE r - | | AVE r - | | |
|---|---|---|---|---|---|---|---|
| | | | correlation | AVE r - | correlation | | AVE |
| | AVE BIAS | AVE STDEV | (test device, | correlation | (reference | AVE | sensitivity |
| | (test device, | (test device, | reference | (test device, | device, | sensitivity | (reference |
| | reference | reference | device, | fluid removed, | fluid removed, | (test device, | device, |
| Cohorts | units Ohms) | units Ohms) | no units) | no units) | no units) | units Ohms/L) | units Ohms/L) |
| 1A | 8.48 | 1.01 | 0.85 | 0.91 | 0.93 | 1.67 | 1.89 |
| 1B | 10.82 | 1.05 | 0.89 | 0.93 | 0.94 | 1.69 | 2.31 |

As is clear from the table, subjects with both CHF and ESRD show a larger average BIAS between test and measurement devices, as well as a larger sensitivity for both test and reference devices, than those with ESRD alone. This is presumably related to these subjects' diagnosis of CHF, which means they typically have larger amounts of thoracic fluids distributed throughout the body. However the table indicates that the overall measurement performance of the test device is essentially the same for the two cohorts.

Using the results from the clinical study, the relationship between the test device's TFC value and the reference device's Z₀ value was evaluated with a repeated-measures model. To perform this analysis, a repeated-measures model (which accounts for correlations between successive points in time with an auto-regressive AR(1) term) was used to fit the data, with: Model A—separate slopes for each subject; and Model B—a common slope for all subjects. The results for the two models was then compared to the fit of the two models using the AIC, which is a measure of the relative quality of a statistical model for a given set of data. The results are as follows:

Model A (separate slopes): AIC=1307.3
Model B (same slope): AIC=1400.9

This indicates that separate, subject-specific slopes should be used to compare changes in impedance throughout the entire thoracic cavity (as measured, e.g., with the reference device) with changes in impedance in an isolated region of the sternum (measured, e.g., with the test device).

To further analyze the data, a similar repeated-measures model was used to investigate the relationship between the test device's TFC value and fluid removed (ΔF). This analysis was conducted similarly to that described above. Specifically, TFC values measured by the test device were modeled as a linear function of: 1) fluid removed; 2) using a subject-specific y-intercept term; and 3) using an autocorrelation term that accounts for the dependence of temporally sequential measurements. The following models were then used to fit data corresponding to all subjects to test the equality of slopes: Model A—separate slopes for each subject; and Model B—a common slope for all subjects. As with the analysis described above, the model with the smallest AIC was chosen as the best fitting model. Results for this analysis are shown below:

Model A (separate slopes): AIC=1636.0
Model B (same slope): AIC=1241.7

This indicates that a single (i.e. common) slope can be used to compare changes in fluid in an isolated region of the sternum with changes in impedance from that same region (measured, e.g., with the test device). More specifically, Model B predicts that each subject starts (at time 0) with a unique TFC value, and that for each liter of fluid removed, the TFC value will increase by approximately 1.5 Ohms, i.e. a sensitivity of 1.5 Ohms/L. This model doesn't account for intra-venous saline introduced into each subject (500 mL) during the dialysis period. When this is accounted for, the sensitivity increases to 1.69 Ohms/L. This is essentially identical to the average sensitivity (1.68 Ohms/L) shown above in Table 2.

To further investigate the above, the percentage change of impedance for both the test (TFC) and reference (Z₀) devices was investigated. Here, the percentage change in impedance was calculated by first pooling all subject-specific data collected during the clinical study, and then determining an average value of both TFC and Z₀ for each 200 mL of fluid removed. The percentage change was the average value of impedance at these 200 mL increments divided by the average value of impedance before dialysis was started. For this calculation, only a few subjects had more than 3 L of fluid removed. Thus the average values of TFC and Z₀ above this level reflect data collected from only a few subjects, whereas the average values below this level reflect data collected from a relatively large number of subjects. For example, the impedance value at 4 L of fluid removed is the average of just 2 samples, while that at 2 L removed is the average of 23 samples.

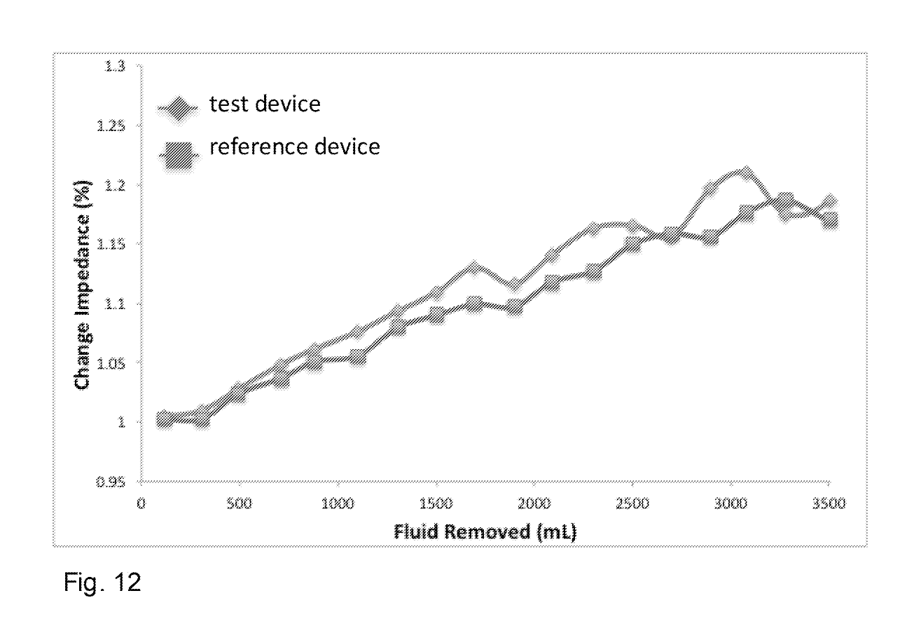

FIG. 12 shows the percentage change of TFC and Z₀ values, calculated as described above, plotted as a function of fluid removed. The curves show nearly identical trajectories, indicating that for dialysis patients the percentage change of fluid measured from a relatively isolated region near the sternum (i.e. TFC) is nearly identical to that measured from the entire thoracic cavity (i.e. Z₀). As described above, the relatively low number of samples at high volumes of fluid removed may explain scatter in the data above 3 L.

Percentage change TFC and Z₀ values from FIG. 12 were than compared directly using standard correlation plots. The plots indicate strong agreement between the two data sets (r²=0.88, r=0.94) and a slope (0.91) near unity, again supporting the claim that the percentage change of fluid measured from the sternum is nearly identical to that measured from the entire thoracic cavity.

Using the sensor as described herein, parameters other than TFC can be detected from patients undergoing hemodialysis, and used to characterize their progression towards decompensation. For example, as shown in FIGS. 13A, B, ECG waveforms measured by the sensor can indicate a normal sinus rhythm (FIG. 13A), or in contrast an abnormal sinus rhythm such as ventricular tachycardia (FIG. 13B). Algorithms operating on the data-analytics engine can analyze the ECG waveforms to detect these excursions, and then notify a clinician using an alarm/alert. This may drive the clinician to pause the dialysis therapy.

Data generated by the sensor may indicate other in-dialysis conditions as well. These include 1) rapid changes in BP leading to hypotension and hypertension; 2) hypoxemia; 3) dysrhythmias; 4) dehydration leading to cramping; 5) chills; 6) nausea; 7) postural changes leading to ineffective therapy; 8) seizures; and 9) rapid blood loss (either internal or external).

In other embodiments, multiple physiological parameters measured by the sensor (e.g. TFC, BP, SV) may be lumped together into a single ‘figure of merit’ or ‘index’, and used to characterize a patient undergoing dialysis.

These and other embodiments of the invention are deemed to be within the scope of the following claims.

What is claimed is:

1. A system for characterizing a patient undergoing a hemodialysis session, comprising:
a body-worn biometric sensor, worn completely on a patient's body on a single location, and comprising: 1) sensing elements for measuring electrocardiogram (ECG), thoracic bio-impedance (TBI), photoplethysmogram (PPG), and phonocardiogram (PCG) waveforms; 2) a processor for collectively analyzing the ECG, TBI, PPG, and PCG waveforms to determine a set of physiological parameters; and 3) a first wireless transceiver configured to transmit the set of physiological parameters;
a gateway system comprising: a second wireless transceiver configured to receive the set of physiological parameters; and
a data-analytics system configured to analyze the set of physiological parameters to determine a status of the patient.

2. A system for estimating a dry weight value of a patient undergoing a hemodialysis session, comprising:
a body-worn biometric sensor, worn on a single location of the patient, and comprising: 1) sensing elements for measuring thoracic bio-impedance (TBI) waveforms; 2) a processor for collectively analyzing the TBI waveforms to estimate a fluid value of the patient, and then estimating the dry weight value of the patient by analyzing the fluid value and a value of the patient's weight before the hemodialysis session begins.

3. A system for characterizing a set of patients undergoing a hemodialysis session, comprising:
a set of body-worn biometric sensors, each sensor configured to be worn on a single location of a patient in the set of patients and comprising: 1) sensing elements for measuring electrocardiogram (ECG), thoracic bio-impedance (TBI), photoplethysmogram (PPG), and phonocardiogram (PCG) waveforms; 2) a processor for collectively analyzing the ECG, TBI, PPG, and PCG waveforms to determine a set of physiological parameters; and 3) a first wireless transceiver configured to transmit the set of physiological parameters; and
a gateway system comprising: a second wireless transceiver configured to receive the set of physiological parameters from each body-worn biometric sensor in the set of body-worn biometric sensors, the gateway system configured to automatically wirelessly pair with and then download a first set of information from a first body-worn biometric sensor in the set, and then once finished automatically wirelessly pair with and then download a second set of information from a second body-worn biometric sensor in the set, the gateway system further configured to repeat this process until sets of information are downloaded from each body-worn biometric sensor in the set of body-worn biometric sensors.

4. A system for estimating a fluid level of a patient undergoing a hemodialysis session, comprising:
a body-worn biometric sensor, worn on a region of the patient proximal to the upper thoracic cavity, and comprising: 1) sensing elements for measuring thoracic bio-impedance (TBI) waveforms for the patient's upper thoracic cavity; and 2) a processor for collectively analyzing the TBI waveforms to determine a fluid value of the patient representing fluid levels in the patient's entire thoracic cavity.

5. A system for characterizing blood pressure values from a patient undergoing a hemodialysis session, comprising:
a body-worn biometric sensor, worn on a single location of the patient, and comprising: 1) sensing elements for measuring electrocardiogram (ECG), thoracic bio-impedance (TBI), photoplethysmogram (PPG), and phonocardiogram (PCG) waveforms; 2) an interface to receive a calibration blood pressure measurement from a cuff-based system; 3) a processor for collectively analyzing the ECG, TBI, PPG, and PCG waveforms and the calibration blood pressure measurement to determine a cuffless blood pressure value; and 3) a first wireless transceiver configured to transmit the cuffless blood pressure value;
a gateway system comprising: 1) a second wireless transceiver configured to receive the cuffless blood pressure value; and
a data-analytics system configured to analyze the cuffless blood pressure value to determine a status of the patient.

6. A sensor for measuring a blood pressure value from a patient, comprising:
a set of four electrodes, with two electrodes in the set connected to an electrical circuit configured to inject electrical current into the patient, and two separate electrodes in the set connected to an electrical circuit configured to sense a voltage from the patient's chest;
an analog system comprising a first analog filter configured to process the voltage to determine an impedance waveform, and a second analog filter configured to process the voltage to determine an ECG waveform; and
a processor configured to process the ECG waveform to determine a first fiducial point, and process the impedance waveform to determine a second fiducial point, and then process a time difference between the first and second fiducial point to determine the blood pressure value;
the sensor worn completely on the patient's body and also comprising a wireless transmitter for transmitting information to an external gateway system.

7. A sensor for measuring a stroke volume value from a patient, comprising:
a set of four electrodes, with two electrodes in the set connected to an electrical circuit configured to inject electrical current into the patient, and two separate electrodes in the set connected to an electrical circuit configured to sense a voltage from the patient's chest;
an analog system configured to process the voltage to determine a thoracic bio-impedance (TBI) waveform;
a sensor configured to measure a phonocardiogram (PCG) waveform; and
a processor configured to process the PCG waveform to determine S1 and S2 heart sounds, and from the time difference between the S1 and S2 heart sounds determine a left ventricular ejection time (LVET), the processor further configured to process the impedance waveform to determine a fiducial point, and then process LVET and the fiducial point to determine the stroke volume value;
the sensor worn completely on the patient's body and also comprising a wireless transmitter for transmitting information to an external gateway system.
